(19) United States
(12) Patent Application Publication
(10) Pub. No.: US 2014/0171282 A1
(43) Pub. Date: Jun. 19, 2014

(54) Process For Making An Absorbent Garment Without Garment Folding

(71) Applicant: KIMBERLY-CLARK WORLDWIDE, INC., Neenah, WI (US)

(72) Inventors: John Timothy Hahn, Merrill, WI (US); Daniel Robert Schlinz, Greenville, WI (US)

(73) Assignee: KIMBERLY-CLARK WORLDWIDE, INC., Neenah, WI (US)

(21) Appl. No.: 13/719,659

(22) Filed: Dec. 19, 2012

(51) Int. Cl. A61F 13/15

(52) U.S. Cl. CPC A61F 13/15707
USPC 493/357

(57) ABSTRACT

A process for making an absorbent garment includes transporting a first web in the machine direction to a point of combination; transporting a second web in the machine direction to the point of combination, wherein the second web is in a substantially superposed relationship to the first web; and folding a first absorbent assembly such that the folded first absorbent assembly has a folded end and an open end. The process further includes disposing the folded first absorbent assembly on one of the first and second webs; attaching the first and second webs to form an envelope at least partially enclosing the folded first absorbent assembly; and cutting the envelope to form a crotch seam.

### BACKGROUND

The present application relates generally to a process for making garments intended to be worn about the lower torso, and particularly to a process for making such a garment including an absorbent assembly.

Garments intended to be worn about the lower torso, such as boxer shorts or other pant-like garments, have a variety of uses including semi-durable garments, disposable garments, and swimwear. Often it can be advantageous for these garments to include an absorbent body; in such configurations, the garments can have applications as training pants, incontinence products, feminine care products, and the like.

Processes for making such garments are known in the art. Nonetheless, the processes that are presently available can often result in garments that are not aesthetically pleasing and that do not provide a comfortable fit. In particular, such processes often do not lend themselves to including an absorbent in the garment. Further, such processes often do not produce garments having a traditional crotch or with hanging legs. Still further, such processes can often be complex, or not be suitable for continuous high-speed converting.

Accordingly, there remains a need for a process that provides garments having traditional garment styling and shape and that includes an absorbent. Further, there is a need for a process that provides such garments with a crotch seam that provides suitable crotch depth. Still further, there is a need for a process for making such garments that is capable of being used in connection with an automated high-speed converting system.

### SUMMARY

In one aspect, the present application is directed to a process for making an absorbent garment, the process defining a machine direction and a cross machine direction, the process including transporting a first web in the machine direction to a point of combination; transporting a second web in the machine direction to the point of combination, wherein the second web is in a substantially superposed relationship to the first web; and folding a first absorbent assembly such that the folded first absorbent assembly has a folded end and an open end. The process further includes disposing the folded first absorbent assembly on one of the first and second webs; attaching the first and second webs to form an envelope at least partially enclosing the folded first absorbent assembly; and cutting the envelope to form a crotch seam.

In another aspect, the present application is directed to a process for making absorbent garments, the process defining a machine direction and a cross machine direction, the process including transporting a first web in the machine direction to a point of combination; transporting a second web in the machine direction to the point of combination, wherein the second web is in a substantially superposed relationship to the first web; and folding a first absorbent assembly such that the folded first absorbent assembly has a folded end and an open end. The process also includes folding a second absorbent assembly such that the folded second absorbent assembly has a folded end and an open end and disposing the folded first and second absorbent assemblies on one of the first and second webs such that the folded edge of the folded first absorbent assembly is adjacent the folded edge of the folded second absorbent assembly. The process also includes attaching the first and second webs to form an envelope at least partially enclosing the folded first absorbent assembly and cutting the envelope between the folded edge of the folded first absorbent assembly and the folded edge of the folded second absorbent assembly to form a crotch seam.

As a result, the present application provides a distinctive process that is capable of continuously producing a garment having an absorbent body, and in which the garment shell is manipulated rather than the absorbent assembly.

The above-mentioned and other aspects of the present application will become more apparent, and the application itself will be better understood by reference to the drawings and the following description of the drawings.

### BRIEF DESCRIPTION OF THE DRAWINGS

The present disclosure will be more fully understood, and further features will become apparent, when reference is made to the following detailed description and the accompanying drawings. The drawings are merely representative and are not intended to limit the scope of the claims.

FIG. 1 representatively illustrates a perspective schematic view of one aspect of a process of the present application; and

FIG. 2 representatively illustrates a perspective schematic view of another aspect of the process of the present application following FIG. 1.

Repeat use of reference characters in the present specification and drawings is intended to represent the same or analogous features or elements of the present disclosure. The drawings are representational and are not necessarily drawn to scale. Certain proportions thereof might be exaggerated, while others might be minimized.

### DETAILED DESCRIPTION

Within the context of this specification, each term or phrase below will include the following meaning or meanings.

“Attach” and its derivatives refer to the joining, adhering, connecting, bonding, sewing together, or the like, of two elements. Two elements will be considered to be attached together when they are integral with one another or attached directly to one another or indirectly to one another, such as when each is directly attached to intermediate elements. “Attach” and its derivatives include permanent, releasable, or refastenable attachment. In addition, the attachment can be completed either during the manufacturing process or by the end user.

“Bond” and its derivatives refer to the joining, adhering, connecting, attaching, sewing together, or the like, of two elements. Two elements will be considered to be bonded together when they are bonded directly to one another or indirectly to one another, such as when each is directly bonded to intermediate elements. “Bond” and its derivatives include permanent, releasable, or refastenable bonding.

“Boxer shorts,” “Shorts,” or “Pants” refers to pants, trunks, briefs, and the like, and include garments that can be relatively loose fitting or snug at the leg area.

“Connect” and its derivatives refer to the joining, adhering, bonding, attaching, sewing together, or the like, of two elements. Two elements will be considered to be connected together when they are connected directly to one another or indirectly to one another, such as when each is directly connected to intermediate elements. “Connect” and its derivatives include permanent, releasable, or refastenable connection. In addition, the connecting can be completed either during the manufacturing process or by the end user.

“Disposable” refers to articles that are designed to be discarded after a limited use rather than being laundered or otherwise restored for reuse.

The terms “disposed on,” “disposed along,” or “disposed toward” and variations thereof are intended to mean that one element can be integral with another element, or that one element can be a separate structure bonded to or placed with or placed near another element.

“Downstream” refers to the positioning of one element or event further in the direction of material travel relative to another element or event in a process.

“Elastic,” “elasticized,” “elasticity,” and “elastomeric” mean that property of a material or composite by virtue of which it tends to recover its original size and shape after removal of a force causing a deformation. Suitably, an elastic material or composite can be elongated by at least 25 percent (to 125 percent) of its relaxed length and will recover, upon release of the applied force, at least 10 percent of its elongation. Desirably an elastic material or composite be capable of being elongated by at least 100 percent (to 200 percent), more desirably by at least 300 percent (to 400 percent), of its relaxed length and recover, upon release of an applied force, at least 50 percent of its elongation.

“Fabrics” is used to refer to all woven, knitted and nonwoven fibrous webs.

“Front-to-back crotch seam” refers to a seam extending from the front region to the back region of a pant-style garment, through the crotch region. The seam can join two separate pieces of material, or separate edges of a single piece of material.

“Hanging legs” refers to the characteristic of a garment intended to be worn about the lower torso where the garment includes material that extends below the crotch of the garment and is intended to generally cover at least a portion of the leg of the wearer; the material can be loose fitting about the leg of the wearer or fit snugly about the leg of the wearer.

“Hydrophilic” describes fibers or the surfaces of fibers that are wetted by the aqueous liquids in contact with the fibers. The degree of wetting of the materials can, in turn, be described in terms of the contact angles and the surface tensions of the liquids and materials involved. Equipment and techniques suitable for measuring the wettability of particular fiber materials or blends of fiber materials can be provided by a Cahn SFA-222 Surface Force Analyzer System, or a substantially equivalent system. When measured with this system, fibers having contact angles less than 90 are designated “wettable” or hydrophilic, while fibers having contact angles greater than 90 are designated “nonwettable” or hydrophobic.

“Layer” when used in the singular can have the dual meaning of a single element or a plurality of elements.

“Liquid impermeable” or “liquid barrier,” when used in describing a layer or multi-layer laminate, means that a liquid, such as urine, will not pass through the layer or laminate, under ordinary use conditions, in a direction generally perpendicular to the plane of the layer or laminate at the point of liquid contact. Liquid, or urine, can spread or be transported parallel to the plane of the liquid impermeable layer or laminate, but this is not considered to be within the meaning of “liquid impermeable” when used herein.

“Machine direction” refers to the direction in which material travels during a production process, as opposed to “cross-machine direction” that refers to the direction that is generally transverse and perpendicular to the machine direction.

“Member” when used in the singular can have the dual meaning of a single element or a plurality of elements.

“Nonwoven” and “nonwoven web” refer to materials and webs of material that are formed without the aid of a textile weaving or knitting process.

“Overlap” refers to the condition where one element is positioned to be at least partially covering another element either directly or indirectly. It should be noted that one element can be beneath the other element and still be overlapping the other element.

“Pants” includes full length and short pants.

“Stretchable” means that a material can be stretched, without breaking, by at least 25% (to 125% of its initial (unstretched) length) in at least one direction, suitably by at least 100% (to 200% of its initial length), desirably by at least 150% (to at least 250% of its initial length).

“Upstream” refers to the positioning of an element or event further in the direction opposite to the direction of material travel relative to another element or event in a process.

These terms can be defined with additional language in the remaining portions of the specification.

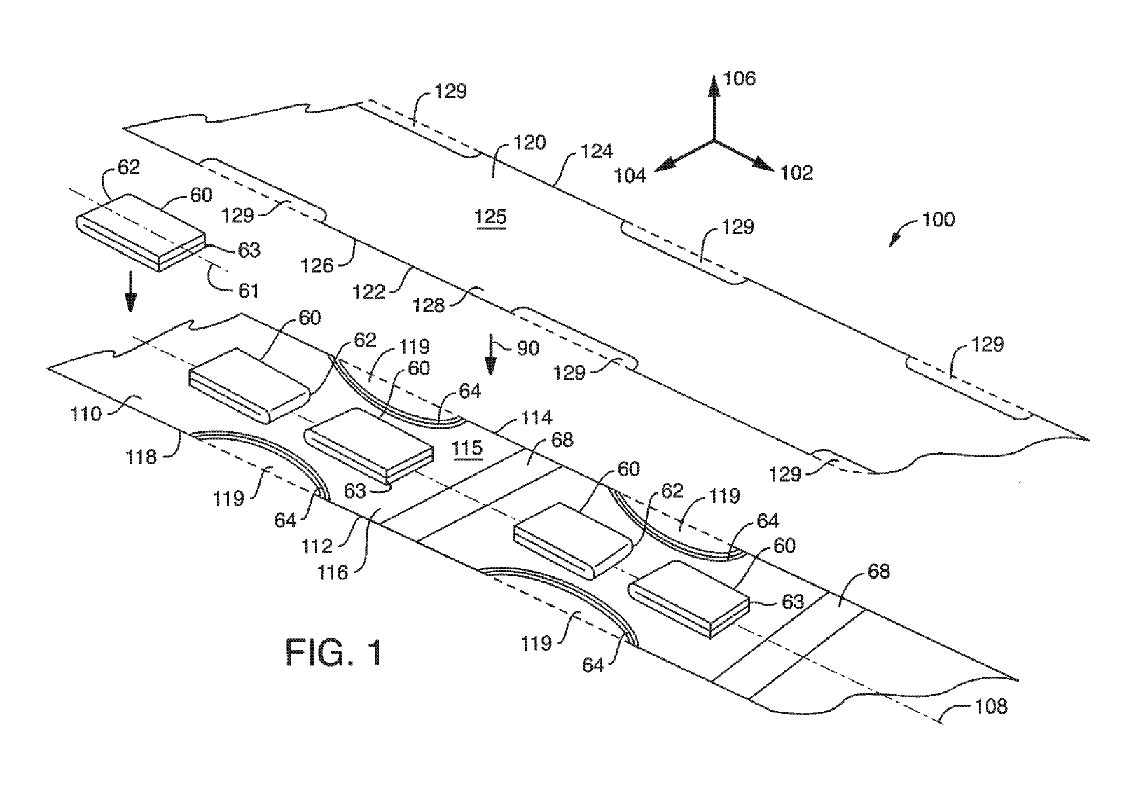

Referring now to the drawings, a process for making garments to be worn about the lower torso is shown in its entirety at reference numeral 100 (FIGS. 1, 2 and 3). The process 100 will be described in terms of making training pants, or pants, but it should be readily recognized that the process of the present application can be equally applicable with pants, trunks, briefs, and other garments that can be worn about the lower torso and having a waist opening, a pair of leg openings, and optionally a pair of hanging legs. Such garments and a process for making them, are described in U.S. Pat. No. 6,192,521 issued Feb. 27, 2001 to Alberts, et al., the disclosure of which is incorporated herein by reference to the extent it is consistent (i.e., not in conflict) herewith.

As representatively illustrated in FIG. 1, the process 100 defines a machine direction indicated at the arrow marked 102, and a cross machine direction indicated at the arrow marked 104 that is perpendicular to the machine direction 102. The process 100 also defines an orthogonal direction, indicated at the arrow marked 106 that is perpendicular to the plane created by the machine direction 102 and the cross machine direction 104. Further, the process 100 also defines a process centerline 108 extending in the machine direction 102.

The process 100 is represented in the illustrated aspects with the orthogonal direction 106 being generally vertical. Nonetheless, as can be readily appreciated by those of skill in the art, the orthogonal direction 106 of the present application can also be generally horizontal or otherwise oriented and still be within the scope of the present application.

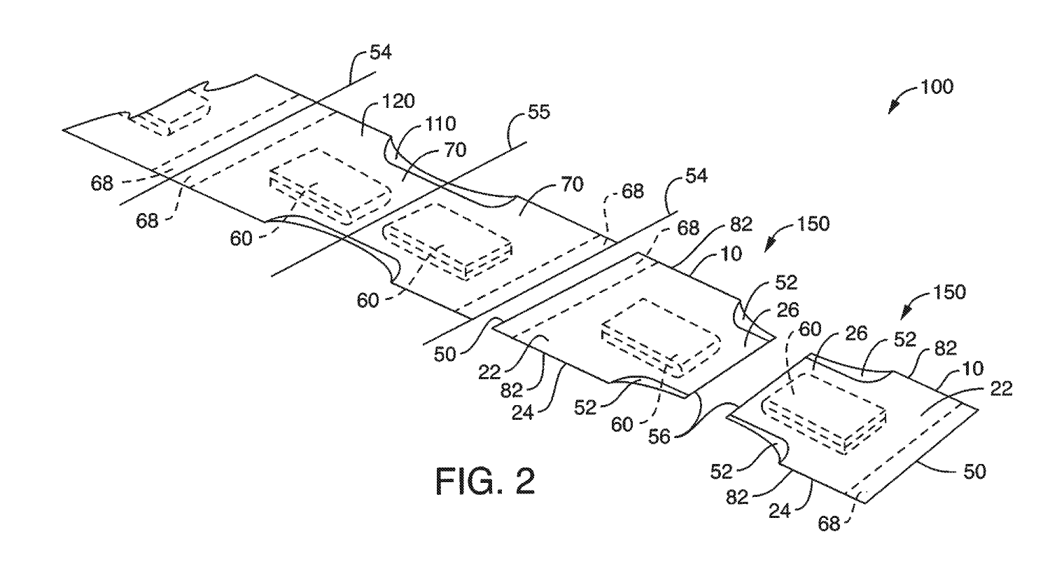

The process includes providing a first web 110 and a second web 120. The first web 110 and the second web 120 can be provided by separate webs or can alternatively be provided by a single web that is folded about the machine direction 102 and then subsequently separated (not shown). As representatively illustrated in FIGS. 1 and 2, the webs 110 and 120 can be provided in a substantially superposed relationship with each other.

The first web 110 defines a first web first edge 112 and a first web second edge 114 that is opposite the first web first edge 112, and a first web interior 115 located within the first web first edge 112 and the first web second edge 114. The first web 110 also defines a first web inner surface 116 that is in facing relationship with the second web 120, and a first web outer surface 118 that is opposite the first web inner surface 116.

The second web 120 defines a second web first edge 122 and a second web second edge 124 that is opposite the second web first edge 122, and a second web interior 125 that is located within the second web first edge 122 and the second web second edge 124. The second web 120 also defines a second web inner surface 126 that is in facing relationship with the first web 110, and a second web outer surface 128 that is opposite the second web inner surface 126. As representatively illustrated in FIGS. 1 and 2, the webs 110 and 120 can be provided in at least a partially facing relationship, and can be in a substantially completely facing relationship. For example, the second web inner surface 126 can be in at least a partially facing relationship with the first web inner surface 116. It should be noted that the first and second web inner and outer surfaces 116, 118, 126, and 128 need not correspond to a body facing surface and an exterior surface of a resultant garment when the garment is produced.

The process 100 is illustrated in FIGS. 1 and 2 as being configured to have the webs 110 and 120 pass through the process 100 in a generally horizontal orientation. Nonetheless, as can be readily appreciated by those of skill in the art, the process 100 can be configured to have the webs 110 and 120 pass through the process 100 in a generally vertical or other orientation and still be within the scope of the present application. Suitably, the webs 110 and 120 can initially be provided to the process 100 with the web edges 112, 114 and 122, 124 in a spaced relationship.

The webs 110 and 120 can be any suitable fabric to provide the absorbent article 10. In particular, the webs 110 and 120 can suitably be of materials that are comfortable against the skin and non-irritating. Since it is contemplated that the absorbent article 10 can be either disposable or durable (i.e., launderable), both nonwoven and woven materials are contemplated for the webs 110 and 120. For example, the webs can be selected from a wide variety of materials, including elastic, stretchable, or nonstretchable materials. Any other type of nonwoven laminate or woven or knitted fabric known to those skilled in the art can also be used. The webs 110 and 120 can be a single layer of material or a multi-layered laminate structure. Moreover, the webs 110 and 120 can each be provided by a plurality of webs attached or otherwise fastened together, or as shown in the illustrated aspects, can each be provided by a single web.

Other suitable materials for the webs 110 and 120 include stretchable nonwovens, non-stretchable nonwovens, and nonwoven laminates including spandex and/or stretchable film. Spandex is any of various elastic textile fibers made chiefly of polyurethane. LYCRA® is a brand of spandex commercially available from E.I. du Pont de Nemours and Company, Wilmington, Del., U.S.A. Alternatively, meltblown laminates are a suitable type of nonwoven laminate. It is desired that the webs 110 and 120 impart a relatively cloth-like texture to the absorbent article 10. The material for the webs 110 and 120 desirably, although not necessarily, has the ability to drape and conform to some extent to the body. In addition, the material can be, but need not be, opaque. Finally, the material for the first web 110 can be the same as the material in the second web 120.

The webs 110 and 120 can be provided by various methods as are known in the art. For example, the webs 110 and 120 can be unwound and drawn through the process 100 via driven rolls, belt conveyors, chain conveyors, and the like or combinations thereof (not shown).

The webs 110 and 120 can be brought together in a facing relationship at a position in the process generally known as the point of combination 90. The webs 110 and 120 can be attached to each other or otherwise combined at the point of combination 90 as described in more detail below.

In the various aspects of the present application, the process 100 can include disposing an absorbent assembly 60 on at least one of the webs 110 and 120. In particular, the absorbent assembly 60 can be disposed upon at least one of the webs 110 and 120 equidistant between the web edges 112, 114 and 122, 124. For example, as representatively illustrated in FIGS. 1 and 2, the absorbent assembly 60 can suitably be disposed on the first web 110. The absorbent assembly 60 can be disposed on at least one of the webs 110 and 120 at various points throughout the process 100 as can be appreciated by one of skill in the art. Suitably, the absorbent assembly 60 can be disposed on at least one of the webs 110 and 120 while the edges 112, 114 and 122, 124 of at least one of the webs 110 and 120 are in a spaced relationship. In particular, the absorbent assembly 60 can be disposed on at least one of the webs 110 and 120 while the webs 110 and 120 are in a planar configuration and thus the edges 112, 114 and 122, 124 are in a spaced relationship. Thus in one aspect, the absorbent assembly 60 can be disposed on the first web 110 while the first web first edge 112 and the first web second edge 114 are in a spaced relationship (FIGS. 1 and 2).

The absorbent assembly 60 can be folded prior to being disposed on one of the webs 110 and 120. Folding can be accomplished by any suitable means. The folded absorbent assembly 60 defines a folded edge 62 and an open edge 63. In the final product, the folded end of the absorbent assembly 60 will generally be disposed in the crotch region of the absorbent article 10, and the open end of the absorbent assembly 60 will be disposed toward the waist opening 50 of the absorbent article 10. The folded absorbent assemblies 60 can be disposed in a uniform manner such that the folded edges 62 of succeeding absorbent assemblies 60 are disposed in the same machine direction 102. The folded absorbent assemblies 60 can also be disposed such that the folded edge 62 of a given absorbent assembly 60 is adjacent the folded edge 62 of the next absorbent assembly 60, and the open edge 63 of that next absorbent assembly 60 is adjacent the open edge 63 of the succeeding absorbent assembly 60.

The absorbent assembly 60 of the present application can define an absorbent assembly centerline 61 that, upon being disposed upon one of the webs 110 and 120, extends in the machine direction 102. More suitably, the absorbent assembly centerline 61 can be parallel, generally parallel, or colinear with the process centerline 108.

The absorbent assembly 60 can be arranged on the webs 110 and 120 in a number of ways, as can be influenced by the configuration of the absorbent assembly 60 and the style of the absorbent article 10. As mentioned above, one of skill in the art can readily contemplate further alternatives depending on the style of the absorbent assembly 60 and the style of the absorbent article 10.

The process 100 of the present application can also include the step of attaching the absorbent assembly 60 to one or both of the webs 110 and 120 in a variety of configurations. As a result, when the absorbent article 10 is completed, the absorbent assembly 60 can be attached to the absorbent article 10 in the front region 22, in the back region 24, or in both regions 22 and 24. Further, the absorbent assembly 60 can be optionally attached to the absorbent article 10 in the crotch region 26 exclusively or while being attached in one or both of the front and back regions 22 and 24. In this way, the process 100 of the present application provides numerous options for attaching the absorbent assembly 60 to the webs 110 and 120. Accordingly, the process 100 of the present application can accommodate the attachment of various absorbent assembly styles, as will be discussed in greater detail below.

The absorbent assembly 60 can be attached to the webs 110 and 120 by a variety of methods as are known in the art. For example, the absorbent assembly 60 can be attached by adhesives, ultrasonic bonding, pressure bonding, sewing, and the like or combinations thereof.

The absorbent assembly 60 can also be releasably attached to the webs 110 and 120, or even refastenably attached to the webs 110 and 120. Such a configuration can be advantageous where the absorbent articles 10 are arranged to be durable or semi-durable, but yet still includes a disposable absorbent assembly 60. For example, the absorbent assembly 60 can be releasably attached to the webs 110 and 120 by using hook and loop fasteners or a cohesive material.

It should be noted that depending on the attachment method, the attachment material (i.e., an adhesive, a mechanical fastener, and the like) can be applied to the absorbent assembly 60, the webs 110 and 120, or both the absorbent assembly 60 and the webs 110 and 120 in order to achieve suitable attachment of the absorbent assembly 60.

After disposing the folded absorbent assembly 60 on one of the webs 110 and 120, the webs 110 and 120 can be brought together and joined in a facing relationship, as illustrated in FIG. 2. The joined webs 110 and 120 form an envelope 70 that partially or completely encloses the absorbent assembly 60. The webs 110 and 120 can be joined by any suitable means, as described in more detail below.

The webs 110 and 120 can be attached to one another with a bonding device (not shown) to provide bond lines and/or seams including a crotch seam 56. In particular aspects, spaced portions of the webs 110 and 120 can be attached to one another at selected locations to provide a plurality of crotch seams 56. The crotch seams 56 can be substantially continuous, or can be provided by a series of intermittent bonds. The crotch seam 56 can be of various shapes to produce the desired result. For example, the crotch seam 56 can be generally rectilinear, curvilinear (for example, circular or oval), generally “D” shaped, or generally “U” shaped. In particular aspects, the crotch seam 56 can be at least partially curvilinear to provide a garment with improved fit and comfort.

The first web 110 and the second web 120 can be attached by the bonding device in various ways as are known in the art. For example, the crotch seam 56 can be formed by bonding the first and second webs 110 and 120 as they travel in the machine direction 102. This bonding can be accomplished by using ultrasonic or thermal bonding wheels rotating in a facing relationship on the outer surfaces 118 and 128 of the webs 110 and 120 to form the crotch seam 56. For example, an anvil wheel and a horn wheel defining a nip can be used to form the crotch seam 56. Alternatively, any suitable bonding method known in the art can be used, such as adhesives, pressure bonding, sewing or the like.

For the crotch seam 56 in particular, bonding the two webs 110 and 120 can include cutting the webs 110 and 120 at the crotch seam 56, where the cutting can be accomplished using the bonder itself or a separate or integrated cutter.

The process 100 can further include the step of separating the webs 110 and 120 into a plurality of discrete individual sections or garment chassis 150 (FIG. 2). The garment chassis 150 would include at least the webs 110 and 120 and the absorbent assembly 60 and can optionally include other components depending upon where in the process 100 the separation occurs.

The webs 110 and 120 can be separated into garment chassis 150 in a variety of ways as are known in the art. For example, the process 100 can include a separation device (not shown) that cuts the webs 110 and 120 into individual garment chassis 150. Specifically, the separation device can be a die cutter, a water cutter, a rotary cutter, an ultrasonic cutter, or the like. Alternatively, the separation device can be a knife and anvil roll that is registered with the process to cut the webs 110 and 120 at a desired waist cut 54 and crotch cut 55 to provide the desired style of garment.

The process 100 of the present application can further include selectively removing portions of the webs 110 and 120 to form cutouts. For instance, as representatively illustrated in FIGS. 1 and 2, the process 100 of the present application can further include selectively removing a portion of the first web 110 to define a first web cutout 119. In addition, the process 100 of the present application can include selectively removing a portion of the second web 120 to define a second web cutout 129. The cutouts 119 and 129 can intersect with the edges of the webs 112, 114, 122, and 124. In one aspect, the cutouts 119 and 129 are generally aligned once the webs 110 and 120 are brought together. In other aspects of the present application, the cutouts 119 and 129 are generally not aligned once the webs 110 and 120 are brought together. In yet other aspects of the present application, the cutouts 119 and 129 are generally differently shaped to account for desired differences in the front and back of the absorbent article 10.

The cutouts 119 and 129 can be any suitable shape to provide openings in the absorbent article 10. For example, the cutouts 119 and 129 can be rectangular, oval shaped, curvilinear, rectilinear, and the like or combinations thereof. In particular, as representatively illustrated in FIGS. 1 and 2, the cutouts 119 and 129 can be elongated in the machine direction 102 and can be at least partially curvilinear.

The cutouts 119 and 129 can be accomplished by various methods as are known in the art. For example, the removal can be achieved by a cutting device (not shown) such as cutting rolls, a die cutting assembly, a water cutting device or an ultrasonic cutter, or combinations thereof. Alternatively, other suitable cutting methods known in the art can be used. The cutouts 119 and 129 can happen to each web 110 or 120 individually or simultaneously, and can happen prior to, concurrent with, or after the webs 110 and 120 reach the point of combination 90.

It should be noted that the attaching of the webs 110 and 120 and the removal of portions of the webs 110 and 120 need not occur in a particular order, and, moreover, need not happen sequentially. For example, the removal can occur prior to the joining of the webs 110 and 120 or alternatively, the joining of the webs 110 and 120 can occur prior to the removal of portions of the webs 110 and 120. In yet another alternative, the joining of the webs 110 and 120 can occur at the same time as removal of portions of the webs 110 and 120. This can be accomplished by utilizing an ultrasonic bonder that is also capable of cutting.

As can be readily appreciated, the waist cut 54 and the crotch cut 55 can be located as necessary in the webs 110 and 120, and can suitably be positioned proximate one another in the webs 110 and 120, or be otherwise associated. For example, as representatively illustrated in FIGS. 1 and 2, the crotch seam 56 can be located adjacent another crotch seam 56.

An advantage of the process 100 described herein is that the absorbent article 10 can be made without the complicated machinery necessary to fold an absorbent article 10. Folding the absorbent assembly 60 and disposing the folded absorbent assembly 60 on one of the webs 110 and 120 prior to bringing the webs 110 and 120 together eliminates the need to fold the absorbent article 10 or either of the webs 110 and 120.

To further enhance containment and/or absorption of body exudates, the absorbent article 10 can also suitably include waist elastics 68 and leg elastics 64, as are known to those skilled in the art. For the purpose of clarity, the leg elastics 64 are not illustrated in FIG. 2. The waist elastics 68 can be operatively joined to one or both of the webs 110 and 120 prior to, concurrent with, or after the webs 110 and 120 are brought together. Similarly, the leg elastics 64 can be operatively joined to one or both of the webs 110 and 120 prior to, concurrent with, or after the webs 110 and 120 are brought together.

The waist elastics 68 and the leg elastics 64 can be formed of any suitable elastic material. As is well known to those skilled in the art, suitable elastic materials include sheets, strands or ribbons of natural rubber, synthetic rubber, or thermoplastic elastomeric polymers. In one particular aspect, for example, the leg elastics 64 can include a plurality of dry-spun coalesced multifilament spandex elastomeric threads sold under the trade name LYCRA® and available from E. I. Du Pont de Nemours and Company, Wilmington, Del., U.S.A.

The webs 110 and 120 can be attached together to form a waist opening 50 and a pair of leg openings 52 to provide the absorbent article 10. In particular, as representatively illustrated in FIGS. 1 and 2, the first web first edge 112 can be attached to the second web first edge 122, and the first web second edge 114 can be attached to the second web second edge 124. Accordingly, the absorbent article 10 with a waist opening 50, a pair of leg openings 52, and optionally a pair of hanging legs is provided.

In a particular aspect, the attaching of the first web first edge 112 to the second web first edge 122 and the first web second edge 114 to the second web second edge 124 can form a pair of side seams 82. The side seams 82 can take any number of forms, including both refastenable and non-refastenable seams as is known in the art. The provision of the side seams 82 can be accomplished in the manner described in U.S. Pat. No. 5,046,272, issued Sep. 10, 1991 to Vogt et al., or in the manner described in U.S. Pat. No. 6,585,840, issued Jul. 1, 2003 to Alberts et al., or in the manner described in PCT Publications WO 01/87562 by Tomsovic, et al., WO 01/87217 by Durrance, et al., WO 01/87753 by Csida et al., and/or WO 01/87218 by Vogt, et al., all of which are incorporated herein by reference to the extent they are consistent (i.e., not in conflict) herewith.

As is known in the art, the side seams 82 can be inward or outward butt seams, welded bead seams, or lap seams, or can be any other seam type as are known in the art. It is contemplated that the side seams 82 can be fastened along only a portion of the distance between the waist opening 50 and the leg openings 52. For instance, the seams 82 can be fastened at the waist opening 50, leaving a slit open above the leg openings 52, such as in the style of some running or athletic garments. Alternatively, the side seams 82 can be fastened from the waist opening 50 to the respective leg openings 52.

Optionally, the process 100 can be configured to provide the absorbent article 10 such that the webs 110 and 120 can be attached together at a different time and/or location to form a pair of side seams 82 and a waist opening 50 and a pair of leg openings 52. That is, the process 100 can be configured to provide an intermediate garment element where the attaching of the first web first edge 112 to the second web first edge 122 and the attaching of the first web second edge 114 to the second web second edge 124 are completed at another location or by the end user. In particular, the edges 112, 114, 122, and 124 can include complementary fasteners, such as hook and loop fasteners, so that the webs 110 and 120 can be attached together at a later time. In such an arrangement, the absorbent article 10 can, for example, be packaged with the edges 112, 114, 122, and 124 unattached so that the end user can attach the webs 110 and 120 and obtain a customized fit.

The process 100 can further include attaching waist elastics 68 to the webs 110 and 120. For example, a portion of waist elastics 68 can be attached to the first web 110 and a separate portion of waist elastics 68 can be attached to the second web 120. The waist elastics 68 can be attached to the webs 110 and 120 in a variety of locations. In a particular aspect and as representatively illustrated in FIGS. 1 and 2, a portion of waist elastics 68 can extend substantially in the cross machine direction 104 from the first web first edge 112 to the first web second edge 114. Similarly, another portion of waist elastics 68 can extend substantially in the cross machine direction 104 from the second web first edge 122 to the second web second edge 124. Suitably, the waist elastics 68 can be attached to the outer surfaces 118 and 128 of the webs 110 and 120.

The waist elastics 68 can be placed on the webs 110 and 120 as necessary to be located proximate the waist opening 50 of the finished absorbent article 10. Moreover, the attaching of the waist elastics 68 can occur at a variety of points during the process 100. Desirably, the waist elastics 68 are positioned upon the webs 110 and 120 so as to be proximate the waist cut 54 upon separation of the webs 110 and 120 into individual garment chassis 150.

The waist elastics 68 can be formed of any suitable elastic material. As is well known to those skilled in the art, suitable elastic materials include sheets, strands or ribbons of natural rubber, synthetic rubber, or thermoplastic elastomeric polymers. In one particular aspect, for example, the waist elastics 68 includes a plurality of dry-spun coalesced multifilament spandex elastomeric threads sold under the trade name LYCRA® and available from E.I. du Pont de Nemours and Company, Wilmington, Del., U.S.A. Alternatively, the elastic material includes a stretch-thermal laminate (STL), a neck-bonded laminated (NBL), a reversibly necked laminate, or a stretch-bonded laminate (SBL) material. Methods of making such STL, NBL and SBL materials are well known to those skilled in the art and described in U.S. Pat. No. 4,663,220 issued May 5, 1987 to Wisneski et al.; U.S. Pat. No. 5,226,992 issued Jul. 13, 1993 to Morman; European Patent Application No. EP 0 217 032 published on Apr. 8, 1987 in the name of Taylor et al.; PCT Publication WO 01/88245 published on Nov. 22, 2001 in the name of Welch, et al.; all of which are incorporated herein by reference to the extent that they are consistent (i.e., not in conflict) herewith.

As mentioned above, the process 100 can include attaching an absorbent assembly 60 to at least one of the webs 110 or 120. The absorbent assembly 60 can be any structure that is generally compressible, conformable, non-irritating to the skin, and capable of absorbing and retaining liquids and certain body wastes. The absorbent assembly 60 can be manufactured in a wide variety of sizes and shapes, from a wide variety of liquid absorbent materials commonly used in the art, and can be stretchable, non-stretchable, or elastic. For example, the absorbent assembly 60 can suitably include a matrix of hydrophilic fibers, such as a web of cellulosic fluff, mixed with particles of a high-absorbency material commonly known as superabsorbent material. In a particular aspect, the absorbent assembly 60 includes a matrix of cellulosic fluff, such as wood pulp fluff, and superabsorbent hydrogel-forming particles. Suitable superabsorbent materials are available from various commercial vendors, such as Dow Chemical Company located in Midland, Mich. U.S.A., and Stockhausen GmbH & Co. KG, D-47805 Krefeld, Federal Republic of Germany. Typically, a superabsorbent material is capable of absorbing at least about 15 times its weight in water, and desirably is capable of absorbing more than about 25 times its weight in water.

In one aspect, the absorbent assembly 60 includes a blend of wood pulp fluff and superabsorbent material. One suitable type of pulp is identified with the trade designation CR1654, available from U.S. Alliance, Childersburg, Ala. U.S.A., and is a bleached, highly absorbent sulfate wood pulp containing primarily soft wood fibers and about 16 percent hardwood fibers.

The absorbent assembly 60 can also incorporate other materials that are designed primarily to receive, temporarily store, and/or transport liquid along the mutually facing surface with absorbent assembly 60, thereby maximizing the absorbent capacity of the absorbent assembly 60. One suitable material is referred to as a surge layer (not shown) and includes a material having a basis weight of about 50 to about 120 grams per square meter (gsm), and including a through-air-bonded-carded web of a homogenous blend of 60 percent 3 denier type T-256 bicomponent fiber including a polyester core/polyethylene sheath and 40 percent 6 denier type T-295 polyester fiber, both commercially available from Kosa Corporation of Salisbury, N.C. U.S.A.

The absorbent assembly 60 can also include a liner material that is intended to face the wearer in use. The liner can be manufactured from a wide selection of web materials, such as synthetic fibers (for example, polyester or polypropylene fibers), natural fibers (for example, wood or cotton fibers), a combination of natural and synthetic fibers, porous foams, reticulated foams, apertured plastic films, or the like. A suitable liquid permeable liner is a nonwoven bicomponent web having a basis weight of about 27 gsm. The nonwoven bicomponent can be a spunbond bicomponent web, or a bonded carded bicomponent web. Suitable bicomponent fibers include a polyethylene/polypropylene bicomponent fiber available from CHISSO Corporation, Osaka, Japan.

The absorbent assembly 60 can also include a suitable outercover intended to face away from the body of the wearer in use. The outercover desirably comprises a material that is substantially liquid impermeable. The outercover can be a single layer of liquid impermeable material, or can be a multi-layered laminate structure in which at least one of the layers is liquid impermeable. A suitable liquid impermeable film for use as liquid impermeable inner layer, or a single layer liquid impermeable outercover, is a 0.025 millimeter (0.75 mil) polyethylene film commercially available from Edison Plastics Company of South Plainfield, N.J., U.S. A. The liquid impermeable material can also be configured to permit vapors to escape from the interior of the absorbent body, while still preventing liquids from passing through the outercover. A suitable “breathable” material is composed of a microporous polymer film or a nonwoven fabric that has been coated or otherwise treated to impart a desired level of liquid impermeability. A suitable microporous film is a PMP-1 film material commercially available from Mitsui Toatsu Chemicals, Inc., Tokyo, Japan, or an XKO-8044 polyolefin film commercially available from 3M Company, Minneapolis, Minn., U.S.A.

In particular aspects, the absorbent assembly 60 is thin to provide a slim, comfortable, non-bulky absorbent article 10. Any suitable thin absorbent assembly can be used, such as for example, the thin absorbent described in WO 02/49565, published Jun. 27, 2002, by Sawyer et al., the disclosure of which is incorporated herein by reference to the extent it is consistent (i.e., not in conflict) herewith.

The absorbent assembly 60 optionally can include a pair of containment flaps (not shown) that are configured to provide a barrier to the transverse flow of body exudates. Suitable constructions and arrangements for the containment flaps are generally well known to those skilled in the art and are described in U.S. Pat. No. 4,704,116 issued Nov. 3, 1987 to Enloe, which is incorporated herein by reference to the extent that it is consistent (i.e., not in conflict) herewith.

In the alternative, a pant-like garment insert could be used for the absorbent assembly 60. For example, the pant-like garment insert suitably includes a body side liner, an outer cover, an absorbent assembly 60 between the body side liner and the outer cover, and side panels. Example of suitable inserts include a training pant, such as HUGGIES® PULL-UPS® Disposable Training Pants, and disposable underpants, such as GOODNIGHTS® Disposable Underpants, both manufactured by Kimberly-Clark Corporation, Neenah, Wis., U.S.A. Other alternative inserts include a cod-piece style insert as described in U.S. patent application Ser. No. 10/750,381 filed Dec. 30, 2003, in the name of Fitton.

In yet another alternative, a pad-type absorbent could be used for the absorbent assembly 60. The pad-type absorbent can be attached in the crotch-region 26 of the absorbent article 10. An example of a suitable pad-type absorbent is a feminine care pad such as KOTEX® Feminine Napkins, KOTEX® LIGHTDAYS® Pantiliners, or an incontinence absorbent pad such as POISE® Feminine Guards and Pads or DEPEND® Guards for Men, all manufactured by Kimberly-Clark Corporation, Neenah, Wis., U.S.A.

As representatively illustrated in FIG. 2, an aspect of an absorbent article 10 produced by the process 100 of the present application can include a front region 22, a back region 24, and a crotch region 26. The absorbent article 10 also defines a waist opening 50. The front region 22 includes the portion of the absorbent article 10 that, when worn, is positioned on the front of the wearer while the back region 24 includes the portion of the absorbent article 10 that, when worn, is positioned on the back of the wearer. The crotch region 26 of the absorbent article 10 includes the portion of the short that, when worn, is positioned between the legs of the wearer and covers the lower torso of the wearer. The front and back regions 22 and 24 are joined together at side seams 82 and the left and right sides of the absorbent article 10 are joined together at the crotch seam 56 to define a three-dimensional short configuration having a waist opening 50 with leg openings 52.

The process 100 of the present application is suitable for producing a series of garments (i.e., absorbent article 10), by transporting the webs 110 and 120 in a machine direction 102 and conducting at least a portion of the process steps described above and as illustrated in the figures.

Accordingly, the present application provides a process 100 that is capable of providing a garment to be worn about the lower torso. In particular, the present application provides a continuous process 100 that readily provides a garment including an absorbent assembly 60, and is capable of being run at elevated process speeds. Thus, the process 100 of the present application is capable of mass producing aesthetically pleasing garments.

As various changes could be made in the above constructions and methods, without departing from the scope of the application, it is intended that all matter contained in the above description and shown in the accompanying drawings shall be interpreted as illustrative and not in a limiting sense.

When introducing elements of the application or the preferred aspect(s) thereof, the articles “a”, “an”, “the” and “said” are intended to mean that there are one or more of the elements. The terms “comprising”, “including” and “having” are intended to be inclusive and mean that there can be additional elements other than the listed elements.

What is claimed is:

1. A process for making an absorbent garment, the process defining a machine direction and a cross machine direction, the process comprising:
transporting a first web in the machine direction to a point of combination;
transporting a second web in the machine direction to the point of combination, wherein the second web is in a substantially superposed relationship to the first web;
folding a first absorbent assembly such that the folded first absorbent assembly has a folded end and an open end;
disposing the folded first absorbent assembly on one of the first and second webs;
attaching the first and second webs to form an envelope at least partially enclosing the folded first absorbent assembly; and
cutting the envelope to form a crotch seam.

2. The process of claim 1, further comprising attaching a portion of the folded first absorbent assembly to one of the first and second webs.

3. The process of claim 1, further comprising folding a second absorbent assembly such that the folded second absorbent assembly has a folded end and an open end; and disposing the folded second absorbent assembly on one of the first and second webs such that the folded edge of the folded first absorbent assembly is adjacent the folded edge of the folded second absorbent assembly, wherein cutting includes cutting the envelope between the folded edge of the folded first absorbent assembly and the folded edge of the folded second absorbent assembly.

4. The process of claim 3, wherein cutting includes attaching the first and second webs at the crotch seam.

5. The process of claim 1, further comprising removing a portion of the first web prior to reaching the point of combination to form a first cutout.

6. The process of claim 5, further comprising removing a portion of the second web prior to reaching the point of combination to form a second cutout.

7. The process of claim 6, wherein the first and second cutouts each have a shape, and wherein the first cutout shape is different from the second cutout shape.

8. The process of claim 6, wherein the first cutout is not aligned with the second cutout at the point of combination.

9. The process of claim 1, wherein the garment is made without folding the first web.

10. The process of claim 1, wherein the garment is made without folding the first or second webs.

11. The process of claim 1, further comprising attaching a waistband to the first or second web prior to reaching the point of combination.

12. The process of claim 1, further comprising attaching a leg elastic to the first or second web prior to reaching the point of combination.

13. The process of claim 1, wherein the first absorbent assembly includes a fluid barrier, an absorbent core, and integrated flaps.

14. The process of claim 1, wherein the first or second web is extensible in the cross machine direction.

15. A process for making absorbent garments, the process defining a machine direction and a cross machine direction, the process comprising:
transporting a first web in the machine direction to a point of combination;
transporting a second web in the machine direction to the point of combination, wherein the second web is in a substantially superposed relationship to the first web;
folding a first absorbent assembly such that the folded first absorbent assembly has a folded end and an open end;
folding a second absorbent assembly such that the folded second absorbent assembly has a folded end and an open end;
disposing the folded first and second absorbent assemblies on one of the first and second webs such that the folded edge of the folded first absorbent assembly is adjacent the folded edge of the folded second absorbent assembly;
attaching the first and second webs to form an envelope at least partially enclosing the folded first absorbent assembly; and
cutting the envelope between the folded edge of the folded first absorbent assembly and the folded edge of the folded second absorbent assembly to form a crotch seam.

16. The process of claim 15, wherein cutting includes attaching the first and second webs at the crotch seam.

17. The process of claim 15, further comprising removing a portion of the first web prior to reaching the point of combination to form a first cutout.

18. The process of claim 17, further comprising removing a portion of the second web prior to reaching the point of combination to form a second cutout.

19. The process of claim 18, wherein the first and second cutouts each have a shape, and wherein the first cutout shape is different from the second cutout shape.

20. The process of claim 18, wherein the first cutout is not aligned with the second cutout at the point of combination.
